(19) United States
(12) Patent Application Publication
(10) Pub. No.: US 2021/0015528 A1
(43) Pub. Date: Jan. 21, 2021

(54) Multiple Drive Screw System and Device

(71) Applicants: Stephen James O'Brien, New York, NY (US); Thomas Zink, San Antonio, TX (US); Jeffrey M. Brittan, Cardiff-by-the-Sea, CA (US); Nicholas Cordaro, Encinitas, CA (US)

(72) Inventors: Stephen James O'Brien, New York, NY (US); Thomas Zink, San Antonio, TX (US); Jeffrey M. Brittan, Cardiff-by-the-Sea, CA (US); Nicholas Cordaro, Encinitas, CA (US)

(21) Appl. No.: 16/931,553

(22) Filed: Jul. 17, 2020

Related U.S. Application Data
(60) Provisional application No. 62/876,298, filed on Jul. 19, 2019.

(51) Int. Cl. A61B 17/86; A61B 17/88

(52) U.S. Cl. CPC A61B 17/8615; A61B 17/8635; A61B 17/8888

(57) ABSTRACT

A surgical screw system and device with a screw head having multiple insertion engagement features. The screw preferably a tubular body having a first end and a closed second end, wherein the head configured to engage with a diiver to advance the screw into the bone. The head of the screw has at least two different insertion features, such as a cross shaped insertion feature and a hexalobe shaped insertion. The screw can be advanced or removed via a driver that mates with the cross shaped or the hexalobe shaped insertion features.

### CROSS-REFERENCE TO RELATED APPLICATION

The present application claims priority to, and the benefit of, U.S. Provisional Application No. 62/876,298, which was filed on Jul. 19, 2019 and is incorporated herein by reference in its entirety.

### FIELD OF THE INVENTION

The present invention relates generally to a multiple drive system for removing a surgical screw during a revision surgery (e.g. knee, hip, angle, spine, etc.), and a related insertion/removal device for engaging and manipulating the multiple drive screw device in vivo. More specifically, the multiple drive screw device is designed to provide a surgeon with additional options for removing a stripped or otherwise damaged screw during a revision surgery, and thus reducing the likelihood of complications such as further injury or damage associated with the removal.

### BACKGROUND

Various types of surgical fasteners are used to engage implants or replacements and other devices to bone. For example, in the spinal field, bone screws are commonly used to attach plates, rods and other types of implants and devices to one or more of a patient's vertebrae. Specifically, bone screws are often used in orthopedic surgery to secure bone sections to one another, or to artificial joints, plates or other structural members to be retained in place. The bone screws, which usually have a head for receiving a driver tool, and a threaded shank portion, are threaded into the patient's bone such as may be encountered in hip, knee, elbow or ankle replacement surgeries.

Due to various factors, the implanted screws may need to be removed in vivo, either during the initial surgery or sometime later occasionally even years later during a revision surgery where the implant has failed, been recalled or other complications have set in. Unfortunately, over time, such bone screws may have been damaged during the initial implantation or can deteriorate over time and may be stripped or broken and are no longer removable using the traditional driver device that was used to initially install the bone screw. This can create complications for the patient during the revision surgery as well as difficulties for the surgical team when attempting the extraction, causing delays and frustration and potentially additional cost to the patient, hospital and insurance company.

Consequently, there is a long felt need in the art for a bone screw device that provides a surgeon with multiple different options for subsequent removal of the bone screw. Thus, if one option for removal fails, a user has additional options for removing the bone screw in vivo. There is also a long felt need in the art for an insertion/removal device for engaging and manipulating the multiple drive screw device in vivo.

The present invention discloses a multiple drive screw device comprising a screw component with a head having multiple insertion characteristics. The screw component comprises a first end and a closed second end when the head is positioned at the first end of the implant or bone and configured to engage with a driver to advance the screw into the bone or withdraw the same. A tip is positioned at the second end of the screw. The head of the screw component further comprises at least two different insertion features or characteristics. Through use of the present invention and tool, the screw component can be advanced or removed via a driver component that mates or engages with at least one of the insertion features or characteristics.

While this specification makes specific reference to a multiple drive screw system and the device for removing a stripped, defective or otherwise damaged bone screw from a patient's bone, it will be appreciated by those of ordinary skill in the art that aspects of the present invention are also equally applicable to other like applications and other such surgical screws and/or removal issues.

### SUMMARY

The following presents a simplified summary in order to provide a basic understanding of some aspects of the presently disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, includes a multiple drive screw system and device that is comprised of a screw component with a head having multiple different insertion/engagement features or characteristics. More specifically, the screw component has a generally tubular body with a first end and a closed second end. The tubular body has a tubular wall which defines a cavity. The thickness of the wall of the cavity in a radial direction is preferably smaller than an inner radius of the wall of the cavity. The tubular body further includes an exterior thread on an exterior surface portion of the tubular wall. A head at the first end is configured to engage with a driver to advance the bone screw into the bone or to withdraw the same from the bone and or implant, and a tip at the second end of the screw. The tubular body, the head and the tip are preferably formed as a single unitary piece or unit.

As a further important feature of the present invention, the head of the screw component further comprises at least two different insertion features, characteristics or drive assist configurations. in one embodiment, the head has a cross shaped insertion feature made from two perpendicular aims which extend to a periphery of the head of the screw and covering most of the diameter of the head. There is also a hexalobe shaped insertion feature disposed within the cross shaped insertion feature. The screw component can be advanced or removed via a driver component that mates with the cross shaped feature, such as through a conventional Phillip's head or alternatively a flat head screw driver component, and/or a mating hexalobe shaped driver component. This redundancy in driver options enables the surgical screw to continue to be useful (i.e., driven into or withdrawn from a bone) even if one of the driver options is no longer available (e.g., because the driver option is stripped or is otherwise damaged). In an alternate embodiment, the driver head may be configured to have both driving characteristics on the single head so that the driver head can engage both insertion characteristics and apply more torque to inserting or removing the screw if multiple portions of the screw head are damaged.

In an alternative embodiment, the present invention may comprise a screw component with a head having a hexalobe insertion feature or characteristic within a hexalobe shaped insertion feature. The head may also comprise a first hexalobe shaped insertion feature or characteristic within a second hexalobe shaped insertion feature along with a reverse thread component. The screw component can then be advanced or removed via a driver component that mates or engages with the outer and/or inner hexalobe shaped insertion features or characteristics. Further, the driver component can also engage the reverse thread component to further engage the screw head and aid in removing the screw from a patient's bone. For example, a medical professional could engage the reverse thread component and torque it, turning the driver component slightly counter-clockwise so as to tighten the engagement of the thread component to the driver component, which would then assist in loosening the screw component from the patient's bone and/or implant.

In yet another alternative embodiment of the present invention, the screw head may include a cross shaped insertion feature that extends across the periphery of the head and covers the major diameter of the top of the head and a hexagon shaped insertion feature or characteristic within the cross shaped insertion feature. A further hexalobe shaped insertion feature may also be provided within the hexagon shaped insertion feature. Therefore, the screw component can he advanced or removed via a driver component that mates or engages with either or both of the cross shaped insertion feature, such as a Phillip's head or flat head screw driver component, or a mating hexagon or hexalobe shaped driver component.

In a further alternative embodiment of the present invention, a screw component may include a head comprising a hexalobe shaped insertion feature or characteristic and a hexagon shaped insertion feature or characteristic on the outside or exterior of the hexalobe shaped insertion feature. The screw component can then be advanced or removed via a driver component that mates with either the outer hexagonal shaped insertion feature or the inner hexalobe shaped insertion feature.

In a yet further exemplary embodiment of the presently described invention, a surgical screw and insertion tool combination, is described and includes a screw with a head portion and a body portion. The head portion has a periphery that defines a diameter and a plane. The head portion has at least first and second insertion features, with the second insertion feature disposed centrally within the first insertion feature and each of the first and second insertion features extending inwardly into the head portion from the plane and each of the first and second insertion features fully contained within the periphery of the head. The body has a first end and a second end, with the first end connected to the head and the second end having a tip, the body having a tubular shape and having a threaded area covering at least a portion of the body and below the head and closer to the tip. The body further having a cavity extending from the head toward the tip. A driver tool that has a first end and a second end, with the driver tool second end having a driving feature to mate with at least one of the first and second insertion features of the head.

In a still further exemplary embodiment of the presently described invention, a method of using a surgical screw and insertion tool combination is presented and incldues the steps of initially providing a surgical screw. The screw has a head portion and a body portion, with the head portion having a periphery defining a diameter and a plane. The head portion has at least first and second insertion features, with the second insertion feature disposed centrally within the first insertion feature and each of the first and second insertion features extending inwardly into the head portion from the plane. Each of the first and second insertion features is fully contained within the periphery of the head. The body has a first end and a second end, with the first end connected to the head and the second end having a tip. The body has a tubular shape and a threaded area covering at least a portion of the body and below the head and closer to the tip. The body further having a cavity that extends from the head toward the tip. Next, an insertion tool is supplied. The insertion tool has a first end and a second end, with the second end having a driving feature to mate with at least one of the first and second insertion features of the head. Then a driving feature is selected to drive the surgical screw into the bone based on an insertion force required for an implant procedure. Finally, the surgical screw is inserted into the bone by turning the insertion tool with the driving feature engaged and mated with one of the first and second insertion features.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1A illustrates a perspective view of the head of a multiple drive screw device with a cross shaped insertion feature around the major diameter of the screw head and a hexalobe shaped insertion feature within the cross shaped insertion feature in accordance with the disclosed structure;

FIG. 1B illustrates a perspective view of the multiple drive screw device of FIG. 1A with a cross shaped insertion feature around the major diameter of the screw head and a hexalobe shaped insertion feature within the cross shaped insertion feature in accordance with the disclosed structure; and

FIG. 2A illustrates a perspective view of a driver with a hexalobe shaped end which can be used to advance or retract the multiple drive screw device of FIGS. 1A and 1B in accordance with the disclosed structure

FIG. 2B illustrates a close-up perspective view of the hexalobe shaped end of the driver of FIG. 2A, which can be used to advance or retract the multiple drive screw device of FIGS. 1A and 1B in accordance with the disclosed structure;

FIG. 3 illustrates a perspective view of an alternative embodiment of the multiple drive screw device with a hexalobe shaped insertion feature around the major diameter of the screw head and a hexalobe shaped insertion feature within the outer hexalobe shaped insertion feature in accordance with the disclosed structure;

FIG. 4 illustrates a perspective view of a further alternative embodiment of the multiple drive screw device with a hexalobe shaped insertion feature extending across the perimeter and covering the major diameter of the screw head and a hexalobe shaped insertion feature within the outer hexalobe shaped insertion feature, as well as reverse threads arounds the external diameter of the screw head in accordance with the disclosed structure;

FIG. 5A illustrates a perspective view of the head of a further alternative embodiment of the multiple drive screw device with a cross shaped insertion feature around the major diameter of the screw head and a hexagon shaped insertion feature within the cross shaped insertion feature and a hexalobe shaped insertion feature within the hexagon shaped insertion feature in accordance with the disclosed structure;

FIG. 5B illustrates a perspective view of the further alternative embodiment of the multiple drive screw device of FIG. 5A with a cross shaped insertion feature around the major diameter of the screw head and a hexagon shaped insertion feature within the cross shaped insertion feature and a hexalobe shaped insertion feature within the hexagon shaped insertion feature in accordance with the disclosed structure; and

FIG. 6 illustrates a perspective view of yet another alternative embodiment of the multiple drive screw device with a hexagon shaped insertion feature extending across the perimeter and covering the major diameter of the screw head and a hexalobe shaped insertion feature within the outer hexagon shaped insertion feature in accordance with the disclosed structure.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

Generally stated, and in one embodiment thereof, the present invention discloses a drive screw device comprising a screw component with a head having multiple insertion or engagement features or characteristics. More specifically, the screw component comprises a general tubular body having a first end and a closed second end. The head, having the insertion or engagement features or characteristics is positioned at the first end and configured to engage with a driver to advance the screw into the bone and or implant or withdraw the same, and a tip is positioned at the second end, opposite of the first end. The head of the screw component further comprises at least two different insertion features or characteristics, such as a cross shaped insertion feature around extending across the periphery and covering the major diameter of the head and a hexalobe shaped insertion feature centrally disposed within the cross shaped insertion feature. Therefore, the screw component can be advanced or removed via a driver component that mates or engages with either the cross shaped or the hexalobe shaped or both. This redundancy in driver options enables the surgical screw to continue to be useful (i.e., driven into or withdrawn from a bone and or implant) even if one of the driver options is no longer available (e.g., because the screw head engagement characteristic is stripped, degraded or is otherwise damaged).

Insertion torque is calculated in Newton centimeters (“Ncm”) for implants refers to the electric energy consumed during the initial tapping or insertion of the screws for the implant. The insertion torque must be carefully selected as it can have a significant impact on the loading forces during the surgery and healing of the patient post-surgery. The surgical screw of the present invention should have an insertion torque of between 15 and 35 Ncm. The screw head will typically have a first tensile strength and the threaded portion on the body will have a second tensile strength. In selecting the drive head characteristics for the screw head, a Phillips type of feature will likely withstand less force than a hex type of feature and may be used with applications lower insertion torque settings. A Phillips head feature while lending itself to manufacturing efficiencies may see increased head failure or the head releasing from the shaft of the screw. By providing more than one screw head feature to enable securement of the surgical screw into the bone, one overcomes the shortfall of providing only a single feature and reduces the risk of screw failure.

In alternative embodiments, the present invention may comprise various other configurations of the screw head. For example, the screw head can have a hexalobe insertion feature within a hexalobe shaped insertion feature. The duplicate hexalobe feature will have a first hexalobe engagement characteristic of a first size and second hexalobe engagement characteristic of a second size. Alternatively, the head can comprise a hexalobe shaped insertion feature within a hexalobe shaped insertion feature along with a reverse thread component. Further, the screw head can comprise a cross shaped insertion feature around extending across the periphery of the screw head and covering the major diameter of the head and a hexagon shaped insertion feature centrally disposed within the cross shaped insertion feature, and, a further hexalobe shaped insertion feature within the hexagon shaped insertion feature. The first and second hexagonal or hexalobal elements haying different dimensions for use with different sized driver or insertion took. Additionally,the screw head can include a hexalobe shaped insertion feature and a hexagon shaped insertion feature on the outside or exterior of the hexalobe shaped insertion feature, or the screw head can comprise any such configuration as is known in the art based on the needs and/or wants of the user. For example, a first hexalobe feature can be disposed at one elevation of the screw head and the second hexalobe feature or characteristic can be disposed at a second elevation of the screw head in such a manner that neither the of the features or characteristics interfere with the other feature or characteristic.

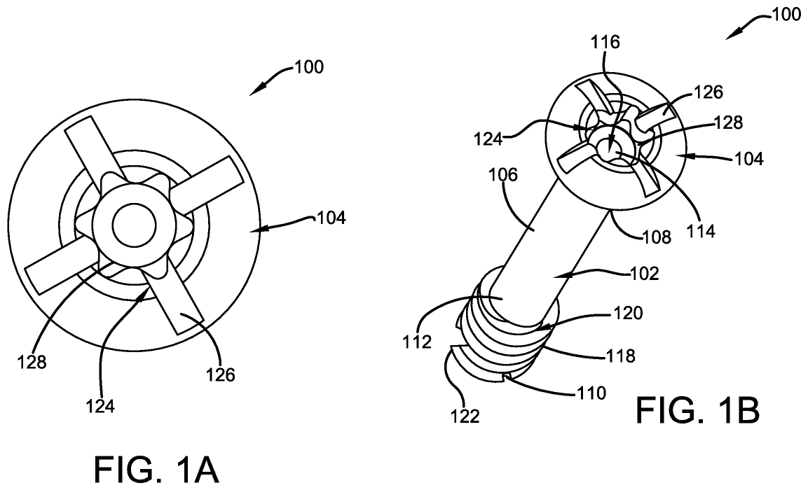

Referring initially to the drawings, FIGS. 1A-B illustrates a drive screw device 100 that has a screw component 102 with a head 104 with multiple insertion features or characteristics. The head has an outer periphery defining a diameter. The head may be flat on top or may be rounded or otherwise rise upward from a plane defining the periphery. The base of the head tapers generally toward the shaft or body of the screw. Both the screw component 102 and the head 104 may be manufactured using typical machining techniques as are known in the art, and are preferably constructed as one unitary part. Notwithstanding the forgoing, the screw component 102 and head 104 are preferably manufactured from titanium, specifically Ti 6 Al 4 V-ELI, though the same can be manufactured from any other suitable medical grade material as is known in the art, including stainless steel, cobalt-chrome alloys, tantalum, other titanium alloys, aluminum and combinations thereof. The screw or any portion of the screw may also have a particle coating ranging from about 80 to over 700 microns to help the screw fuse with the bone and promote bone growth. Such exemplary coatings may include hydroxylapatite (HA), titanium plasma spray (TPS) rough coating, TPS porous coating, resorbable blast media (RBM), anti-wear and sintered coatings.

Additionally, the multiple drive screw device 100 and its various components can be any suitable size, shaped, and/or configuration as is known in the art without affecting the overall concept of the invention. One of ordinary skill in the art will appreciate that the shape, size and configuration of the various potential embodiments of multiple drive screw device 100 as shown in FIGS. 1 and 3-6 are for illustrative purposes only, and that many other shapes, sizes and/or configurations of the multiple drive screw device 100 are well within the scope of the present disclosure. Although dimensions of the multiple drive screw device 100 (i.e., length, width, and height) are important design parameters for good performance, the multiple drive screw device 100 may be any shape, size or configuration that ensures optimal performance during use. The screw is sized for the particular implant or procedure to be performed. For example a hip implant may require a larger screw than an ankle or knee implant.

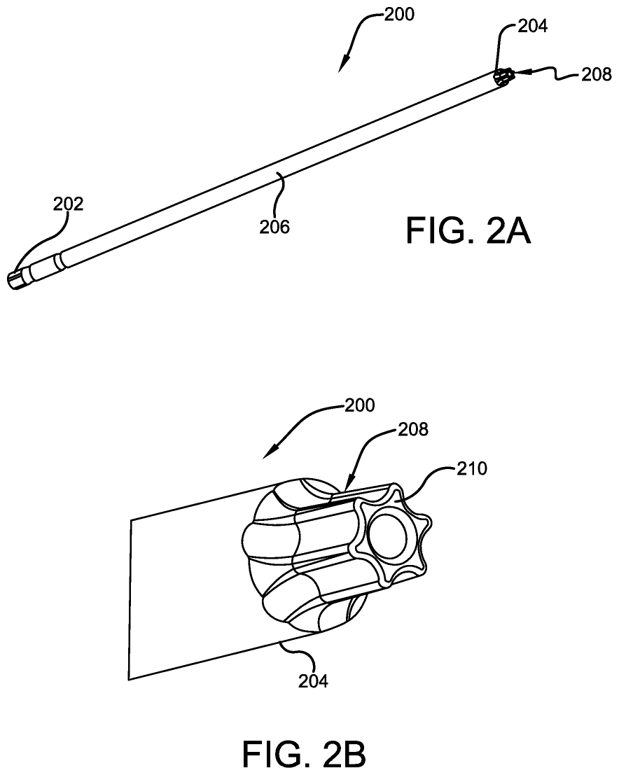

Returning to FIGS. 1A and 1B, the screw component 102 of the multiple drive screw device 100 comprises a tubular body 106 having a first end 108 which is attached to the screw head 104 and a closed second end 110 which may be tapered or have a diameter less than a diameter of the tubular body 106. Further, the tubular body 106 has a generally tubular wall 112 that defines an inner cavity 114 wherein the thickness of the exterior tubular wall 112 in a radial direction is larger than an inner radius 116 of the cavity 114. The tubular body 106 further includes an exterior thread 118 portion on an exterior tubular surface portion 120 of the tubular wall 112, a head 104 at the first end 108 configured to engage with a driver (see e.g., FIGS. 2A and 2B) to advance/withdraw the bone screw component 102 into/Born a patient's bone, and/or implant and a tip 122 at the second end 110. The tip 122 helps aid in the insertion and positioning of the screw component into a patient's bone (not shown). As stated previously, the tubular body 106, the head 104, threads 118 and the tip 122 are preferably formed as a single unitary device. The threads 118 on the exterior of the tubular body may cover only a portion of the tubular body 106 or may cover the entire length of the body stretching from beneath the head 104 to the tip 122.

Additionally, the head 104 of the screw component 102 further comprises at least two different insertion features or drives 124, though more than two insertion features 124 can be utilized depending on the preferences of a user, design of the implant or requirements of a procedure. The insertion features or characteristics 124 are preferably indentations located across the diameter of the head 104 and within the periphery for receipt of a portion of a driver or insertion device as described more fully below, though it is also contemplated that protrusions (i.e. elements that protrude outwardly from head 104) could also be used and the associated driver device modified accordingly to engage the extended feature. The insertion features 124 are preferably any combination of hexagon, hexalobe, or cross shaped configurations to ensure a secure and tight fit with the drive portion or head of the driver, though other geometric and non-geometric shapes may also be used provided that they correspond to the shape and size of the end of the driver, and more importantly provide enough gripping engagement between the driver and screw to enable full insertion of the screw into the bone and or implant device. Additionally, the insertion features 124 can include multiple shapes layered within each other, such as having a hexalobe on the inside of the head 104 and a hexagon around the major diameter of the head 104, or vice versa. In addition, the hexalobe and hexagon shapes can appear at different planes of the device, one above the other or slightly offset from one another.

FIG. 2A illustrates a perspective view of a driver 200 with a hexalobe shaped end which can be used to advance or retract the multiple drive screw device of FIGS. 1A and 1B in accordance with the disclosed invention. More specifically, driver 200 is preferably comprised of a first end 202, a second or inserter end 204, and an elongated, generally cylindrical body member 206 positioned therebetween. The first end 202 may be in the shape of a handle for easy manipulation/rotation of the driver 200, and the inserter end 204 is meant to engage one or more of the insertion characteristics or features 124 of the head 104. More specifically, and as best shown in FIG. 2B, an exemplary inserter end 204 of driver 200 preferably comprises one or more male components 208 shaped as, for example, a hexagon, or hexalobe 210. The driver end 204 may have other driver features such as a cross shaped, or any other suitable geometric or non-geometric shape as is known in the art, or reverse threads, etc. which accommodate the insertion characteristics or features on the head of the screw to be used for insertion.

For example, as shown in FIGS. 1A-B, the screw head 104 is depicted as having a cross shaped insertion feature 126 extending across the periphery of the head and around the major diameter of the head 104 and a hexalobe shaped insertion feature 128 centrally disposed within the cross shaped insertion feature 126. Therefore, the screw component 102 can be advanced or removed via a driver component that mates with the cross shaped feature 126, such as a s head or flat head screw driver component, or a mating hexalobe shaped 128 driver component or other features that may be resident in the design of the head.

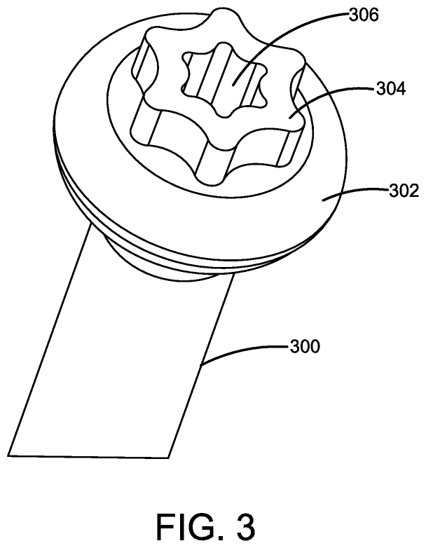

FIG. 3 illustrates a perspective view of an alternative embodiment of the multiple drive screw device 300 with a hexalobe shaped insertion feature extending around and inside the periphery of the screw head and covering the major diameter of the screw head and a hexalobe shaped insertion feature within the outer hexalobe shaped insertion feature in accordance with the disclosed invention. The inner hexalobe feature and the outer hexalobe features both have a top surface in a similar plane, but he inner hexalobe feature may extend below the lower end of the outer hexalobe feature and into the cavity of the screw. In this way, a longer driver head can extend into the cavity providing significantly more driving surface to engage the screw with as opposed to having the screw head features being coterminous with the screw head. More specifically, screw component 300 has a head 302 having a first hexalobe shaped insertion feature 304 around the major diameter of the head 302, and a second hexalobe shaped insertion feature 306 within the first hexalobe shaped insertion feature 304. Therefore, the screw component 300 can be advanced or removed via a driver component that mates with the hexalobe shaped insertion feature (304 or 306) via either a male hexalobe shaped end or a female hexalobe shaped end.

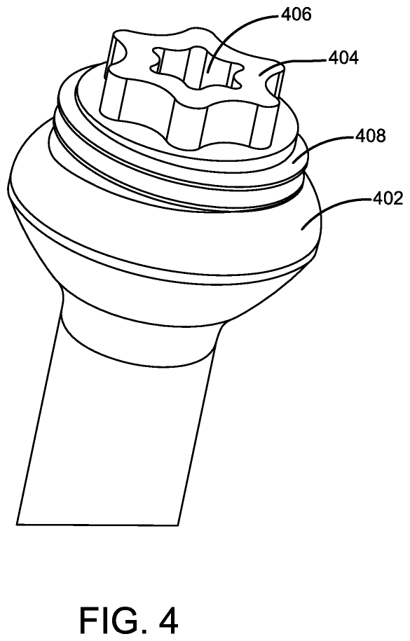

FIG. 4 illustrates a perspective view of a further alternative embodiment of the multiple drive screw device 400 with a hexalobe shaped insertion feature around the major diameter of the screw head and extending inwardly of a periphery of the screw head and a hexalobe shaped insertion feature within the outer hexalobe shaped insertion feature. In this embodiment, the screw head is also provided with reverse threads around the external diameter of the screw head in accordance with the disclosed structure. Each of the reverse threads and hexalobe features extends upwardly from the screw head base. More specifically, screw component 400 has a head 402 that comprises a first hexalobe shaped insertion feature 404 around the major diameter of the head 402 and a second hexalobe shaped insertion feature 406 within the first hexalobe shaped insertion feature 404 along with a reverse thread component 408, also around the major diameter of the head 402 and positioned outwardly from insertion feature 404. Each of the insertion feature 404 and reverse thread component 408 extend generally inwardly from the periphery of the screw head. The screw component 400 can then be advanced or removed via a driver component that engages with the outer or inner hexalobe shaped insertion features (404 and/or 406) or both if a design requires additional torque power to chive the scree into the bone and or implant. Further, the driver component can also engage the reverse thread component 408 to further engage the screw component 400, and aid in removing the screw component 400 from a patient's bone. When a medical professional engages the reverse thread component 408 and torque it, turning the driver component counter clockwise so as to tighten the thread component 408 to the driver component, which would then loosen the screw component 400 as the driver is become locked in position on the head of the screw. This additional locking feature ensures that the driver head does not disengage from the screw during the removal or extraction.

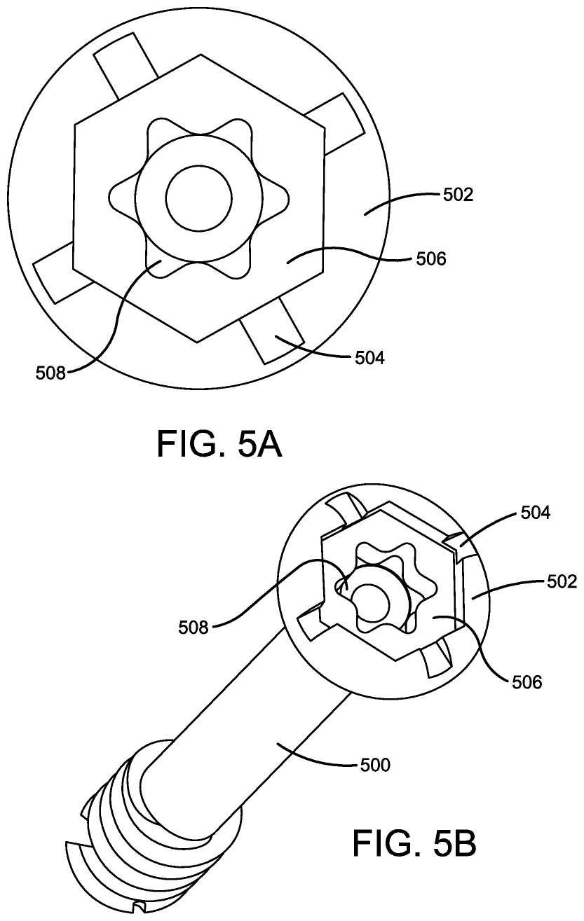

FIG. 5A illustrates a perspective view of the head of a further alternative embodiment of the multiple drive screw device 500 with a cross shaped insertion feature around the major diameter of the screw head, and positioned inwardly of the periphery of the screw head and a hexagon shaped insertion feature centrally disposed within the cross shaped insertion feature. The screw head of this presently described embodiment further includes a hexalobe shaped insertion feature within the hexagon shaped insertion feature in accordance with the disclosed structure. More specifically, screw component 500 has a head 502 with a cross shaped insertion feature 504 around the major diameter of the head 502, a hexagon shaped insertion feature 506 within the cross shaped insertion feature 504, and a hexalobe shaped insertion feature 508 within the hexagon shaped insertion feature 506. Therefore, the screw component 500 can be advanced or removed via a driver component that mates or engages with the cross shaped insertion feature 504, such as a Phillip's head or flat head screw driver component or a mating hexagon 506 or hexalobe shaped 508 driver component or some combination of the three engagement features or characteristics.

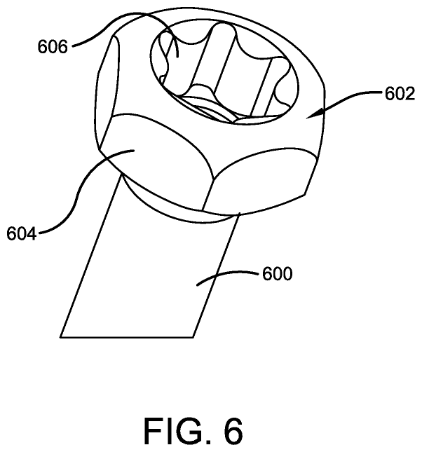

FIG. 6 illustrates a perspective view of yet another alternative embodiment of the multiple drive screw device 600 with a hexagon shaped insertion feature around the major diameter of the screw head and forming the periphery of the screw head and a hexalobe shaped insertion feature centrally disposed within the outer hexagon shaped insertion feature in accordance with the disclosed structure. More specifically, screw component 600 has a head 602 having a hexagon shaped insertion feature 604 around the major diameter or periphery of the head 602 and a hexalobe shaped insertion feature 606 within the hexagon shaped insertion feature 604. Therefore, the screw component 600 can be advanced or removed via a driver component that mates with the hexagon shaped insertion feature 604 or the hexalobe shaped insertion feature 606 or both of the features or characteristics to provide increased torque for the removal or withdrawal of the screw head.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A surgical screw comprising:
a screw having a head connected to a body, the head tapering from a first diameter to a second diameter, the second diameter equal to a diameter of the body, the head having a periphery defining an inner diameter, and
the head having a first insertion feature inside the periphery of the head and extending across the diameter of the head and a second insertion feature centrally disposed within the first insertion feature, with the first and second insertion features each haying a different shape and configuration.

2. The surgical screw of claim 1, wherein the body has a first end and a second end the first end connected to the head and the body having an exterior wall, the body further including a threaded portion covering at least a portion of the exterior wall.

3. The surgical screw of claim 2, wherein the body includes a cavity formed within the body, the cavity extending from the first end into a length of the screw and towards the second end.

4. The surgical screw of claim 2, wherein the body includes an exterior thread on at least a portion of the exterior wall of the body.

5. The surgical screw of claim 2, wherein at least one of the head and body has a coating selected from a group including hydroxylapatite (HA), titanium plasma spray (TPS) rough coating, TPS porous coating, resorbable blast media (RBM), anti-wear and sintered coatings.

6. The surgical screw of claim 1, wherein the first insertion feature is selected from a group including a hexagon shape, a hexalobe shape, or a cross shaped insertion feature.

7. The surgical screw of claim 1, wherein the second insertion feature is selected from a group including a hexagon shape, a hexalobe shape, or a cross shaped insertion feature.

8. The surgical screw of claim 2, wherein the surgical screw can withstand torque insertion forces of between 15 and 35 Ncm.

9. The surgical screw of claim 1, wherein the second insertion feature extends inwardly of the head a distance further than the first insertion feature.

10. The surgical screw of claim 1, wherein the second insertion feature can withstand a higher torque insertion force than the first insertion feature.

11. The surgical screw of claim 1, wherein the second insertion feature is a hex shaped feature and the first insertion feature is a Phillips or cross shaped feature.

12. The surgical screw of claim 2, wherein the head includes a threaded locking feature having a threading direction opposite that of the threaded portion.

13. A surgical screw and insertion tool combination, comprising;
a screw having a head portion and a body portion, the head portion having a periphery defining a diameter and a plane, the head portion having at least first and second insertion features, with the second insertion feature disposed centrally within the first insertion feature and each of the first and second insertion features extending inwardly into the head portion from the plane and each of the first and second insertion features fully contained within the periphery of the head;
the body having a first end and a second end, the first end connected to the head and the second end having a tip, the body having a tubular shape and having a threaded area covering at least a portion of the body and below the head and closer to the tip, the body further having a cavity extending from the head toward the tip; and
a driver tool having a first end and a second end, the driver tool second end having a driving feature to mate with at least one of the first and second insertion features of the head.

14. The surgical screw and insertion tool combination as recited in claim 13, wherein the driving features mates with each of the first and second insertion features of the head.

15. The surgical screw and insertion tool combination as recited in claim 13, wherein the head has a third insertion feature distinct from each of the first and second insertion features.

16. The surgical screw and insertion tool combination as recited in claim 13, wherein the head has a threaded locking feature with threads running in an opposite direction to that of the threaded portion on the body of the surgical screw.

17. The surgical screw and insertion tool combination as recited in claim 13, wherein the first and second insertion features can withstand torque insertion forces of between 15 and 35 Ncm and at least one of the head and body has a coating selected from a group including hydroxylapatite (HA), titanium plasma spray (TPS) rough coating, TPS porous coating, resorbable blast media (RBM), anti-wear and sintered coatings.

18. A method of using a surgical screw and insertion tool combination, comprising the steps of;
providing a surgical screw having a head portion and a body portion, the head portion having a periphery defining a diameter and a plane, the head portion having at least first and second insertion features, with the second insertion feature disposed centrally within the first insertion feature and each of the first and second insertion features extending inwardly into the head portion from the plane and each of the first and second insertion features fully contained within the periphery of the head, and the body having a first end and a second end, the first end connected to the head and the second end having a tip, the body having a tubular shape and having a threaded area covering at least a portion of the body and below the head and closer to the tip, the body further having a cavity extending from the head toward the tip;
supplying an insertion tool, the insertion tool having a first end and a second end, the insertion tool second end having a driving feature to mate with at least one of the first and second insertion features of the head;
selecting a driving feature to drive the surgical screw into the bone based on an insertion force required for an implant procedure; and
inserting the surgical screw into the bone by turning the insertion tool with the driving feature engaged and mated with one of the first and second insertion features.

19. A method of using a surgical screw and insertion tool combination as recited in claim 18, including a further step of removing the surgical screw after the step of insertion by selecting one of the driving features and using a threaded locking feature disposed on the head, with the locking feature having threading in a direction different than the threaded area of the body.

20. A method of using a surgical screw and insertion tool combination as recited in claim 18, wherein the first and second insertion features can withstand torque insertion forces of between 15 and 35 Ncm and at least one of the head and body has a coating selected from a group including hydroxylapatite (HA), titanium plasma spray (TPS) rough coating, TPS porous coating, resorbable blast media (RBM), anti-wear and sintered coatings.
